(19) United States
(12) Patent Application Publication
(10) Pub. No.: US 2024/0361418 A1
(43) Pub. Date: Oct. 31, 2024

(54) SYSTEMS AND METHODS FOR CALIBRATING A DISTANCE MODEL USING ACOUSTIC DATA

(71) Applicant: Eaton Intelligent Power Limited, Dublin 4 (IE)

(72) Inventors: Neil BROCKETT, Kerry (IE); Padhraig RYAN, Kilkenny, Keatingstown (IE)

(21) Appl. No.: 18/256,856

(22) PCT Filed: Dec. 10, 2021
(86) PCT No.: PCT/EP2021/085172

§ 371 Date: Jun. 9, 2023

Related U.S. Application Data
(60) Provisional application No. 63/125,795, filed on Dec. 15, 2020.

(51) Int. Cl. G01S 5/00; G01S 5/02; H04L 67/12; H04W 4/90

(52) U.S. Cl. CPC G01S 5/018; G01S 5/021; G01S 5/0284; H04L 67/12; H04W 4/90

(57) ABSTRACT

Systems and methods for calibrating a distance model and estimating a distance between one or more wireless devices, such as one or more Bluetooth or other types of wireless devices, for social distancing and contact tracing are described. The system includes at least one processor configured to determine an environmental fingerprint from an acoustic signal propagating within an environment, such as a room or another area. The environmental fingerprint may be compared to a database of predefined environmental fingerprints and one or more similar predefined environmental fingerprints identified. The predefined environmental fingerprints may be associated with one or more distance model parameters, which may be used to calibrate a distance model. In addition, the predefined environmental fingerprints may be associated with pretrained distance models. The calibrated or pretrained distance models may be used to process wireless signals in the environment to estimate the distance between users of wireless devices.

### BACKGROUND OF THE DISCLOSURE

The field of the disclosure relates generally to estimating distance between persons to facilitate contact tracing, and more specifically, to systems and methods for calibrating a distance model using acoustic data, and then using the calibrated distance model to estimate distances between users of wireless devices.

A variety of safety protocols and safety equipment, such as personal protective equipment (PPE) exist and may be required by a host of healthcare, industrial, utility, and trade workers to provide a degree of protection from known risks and other hazardous factors. When PPE is utilized in conjunction with appropriate safety protocols, worker safety may be enhanced in otherwise hazardous environments and circumstances.

Challenges remain, however, in effectively overseeing proper adherence by personnel to safety protocols as well as in ensuring appropriate use of PPE during adherence to required safety protocols. For example, it will be appreciated, that employee adherence to certain safety protocols, such as safety protocols requiring a minimum separation or distance between employees (e.g., “social distancing protocols”) can become very important under some circumstances, such as during the spread of an epidemic and/or a global pandemic (e.g., COVID-19), as the case may be. The importance of suitable contact tracing protocols can also dramatically increase in these situations.

While conscientious and well-trained workers will follow PPE and other social distancing protocols, occasional carelessness and mistakes can be expected, with potentially severe consequences. Also, the personal wellness of workers may contribute to carelessness and mistakes by certain workers. In some cases, personal wellness may be part of the safety protocols in place to discourage unhealthy employees from performing certain tasks. An ill worker may lack the same focus as a healthy worker or be subject to distractions that do not ordinarily exist in performing a hazardous task, but to some extent the personal wellness of workers is entirely subjective and workers may not be cognizant of health issues or may overestimate their ability to overcome such issues. Achieving a healthy workforce, and compliance with applicable PPE and social distancing protocols, is therefore an ongoing concern from the safety perspective, and intentional or unintentional violations of social distancing and other proximity protocols can often be difficult to detect, particularly with a large workforce.

At least some computer-implemented monitoring systems exist for monitoring the health and safety of workers, but many such systems lack the capability to detect proximity issues, such as social distancing, contact tracing, and other wellness issues, associated with particular individuals in the group. For example, at least some known PPE monitoring systems lack the capability to determine whether social distancing protocols are adequately followed by members of a workforce absent direct supervision and/or other rudimentary monitoring processes.

In addition, inasmuch as some rudimentary systems may attempt to estimate a distance between a pair of wireless devices, these systems are typically only capable of providing a very coarse estimation of distance, particularly as a result of the tendency of many types of wireless signals, including Bluetooth signals, to vary dramatically in signal strength within a given area. Improvements are therefore desired.

### BRIEF DESCRIPTION OF THE DRAWINGS

Non-limiting and non-exhaustive embodiments are described with reference to the following Figures, wherein like reference numerals refer to like parts throughout the various views unless otherwise specified.

FIG. 1 is a schematic diagram of an example embodiment of a health and safety monitoring system, which may be used to determine proximity of members of a workforce in real-time and based upon one or more acoustic signals and/or one or more other wireless signals, such as one or more BLUETOOTH signals.

FIG. 2 is a schematic diagram of an example embodiment of an electronic computing device, such as proximity detection computing device, that may be used in the health and safety monitoring system shown in FIG. 1.

FIG. 3 is a simplified system diagram illustrating an example embodiment of a signal processing portion of the health and safety monitoring system shown in FIG. 1.

FIG. 4 is a schematic diagram of a server computing device that may be used in the health and safety monitoring system shown in FIG. 1.

FIG. 5A is a first segment of a flowchart illustrating an example embodiment of a process that may be implemented by the health and safety monitoring system shown in FIG. 1 and FIG. 3.

FIG. 5B is a second segment of the flowchart shown in FIG. 5A.

### DETAILED DESCRIPTION OF THE DISCLOSURE

In order to understand the inventive concepts described below to their fullest extent, set forth below is a discussion of the state of the art and certain longstanding problems pertaining to personal wellness and PPE compliance, followed by systems and methods addressing longstanding problems in the art.

It is a practical reality, in certain industries, that exposure of at least some workers to hazardous, or potentially hazardous, working conditions cannot be avoided. As one example, workers in the electrical industry, and more specifically those working in and around electrical power systems, must be trained in the appropriate use of PPE to mitigate possible electrical hazards with which they may be faced.

Aside from hazards associated with electrical shock and electrocution, electrical arc flash incidents are of particular concern. Electrical arcing, or current flow between two or more separated energized conductors, may be experienced when installing, servicing, and maintaining electrical systems. Arcing may occur from electrical fault conditions and can release significant amounts of concentrated radiant energy at the point of arcing in a fraction of a second, resulting in high temperatures that may burn persons exposed to them. Additionally, arcing conditions may produce pressure blasts that are more than sufficient to knock nearby workers off their feet, and shrapnel may be generated by the blast.

Arcing in an electrical power system may suddenly arise in various scenarios that cannot be reliably predicted. For example, insulation failure of components used in electrical systems, including but not limited to cables that interconnect electrical components and equipment may precipitate arcing, as well as a build-up of dust, impurities and corrosion on insulating surfaces. Sparks generated during operation of circuit breakers, during replacement of fuses, and closing electrical connections on faulted lines may also produce an arc. Damage to components and equipment from rodents and pest infestations may result in arcing conditions. Finally, arcing may be the result of unpredictable scenarios of human error such as dropping a tool onto energized conductors, accidental or incidental contact with energized components or equipment, and improper work procedures or mistake in following a procedure to completing a task.

Accordingly, PPE that is adequate or sufficient to provide at least a minimum level of protection to persons against potential electrical hazards has been developed for practically the entire human body, such as for example, electric shock, arc flash and arc blast. Persons wearing such personal protective equipment may be reasonably protected from incidental contact with energized conductors and potentially hazardous arc flash incidents and such PPE may avoid or reduce the likelihood of serious injury if such an arc flash incident occurs. Examples of PPE items may include a hard hat, a face shield, a flame resistant neck protector, ear protectors, a Nomex™ suit, insulated rubber gloves with leather protectors, and insulated leather footwear. Insulated tools may also be provided to complete certain tasks. Such personal protective equipment may be fabricated from various materials to provide, among other things, thermal insulation protection to prevent severe burns to human flesh during high temperature arcing conditions, and to mitigate pressure blasts and shrapnel to avoid life-threatening wounds to a worker's head and torso if arcing conditions were to occur. Different grades of PPE are available to protect against varying degree of risk presented. For example, in the case of electrical fuses that need replacement under energized circuit conditions, fuses of higher electrical ratings may pose a greater risk than fuses of lower electrical ratings, and different amounts or types of personal protective equipment may be required for replacing one fuse, for example, than for replacing another fuse.

Similar considerations exist for other types of hazardous environments rendering similar PPE items desirable for use such as, for example only, petroleum refineries, petrochemical plants, grain silos, wastewater and/or treatment facilities, or other industrial facilities in which sustained or volatile conditions in the ambient environment may be present and may present a heightened risk of fire or explosion and/or a potential exposure to caustic chemicals and substances. Various different grades of PPE are available, which may be similar to or different from the grades of PPE designed for electrical hazards, to meet different risks posed by different situations.

In the healthcare environment, PPE items have been used to protect doctors and nurses in the treatment of patients having conditions that present health risks to healthcare providers when performing certain procedures. Different grades of PPE are available to meet different risks posed by different healthcare procedures. Paramedics, Emergency Medical Technicians (EMTs), Law Enforcement Offices, Firefighters and other emergency responders, as well as military personnel also have PPE items and protocols for responding to certain situations.

Wherever needed, PPE items are subject to appropriate and detailed safety protocols defining their use. Such protocols may detail specific items of PPE (e.g., protective suit, faceshield, gloves, etc.) needed for certain environments or for certain tasks within such environments, processes for obtaining the proper grade of PPE where multiple grades are available, processes for when such PPE items are required to be worn, processes for how such PPE items must be adorned and used, and processes for how PPE should be removed and cleaned for subsequent use. A number of practical challenges exist, however, in effective oversight of the proper use of PPE by personnel in a hazardous environment. Conscientious and well-trained workers will dutifully follow PPE protocols, but occasional misunderstanding, carelessness, and mistake may nonetheless occur with potentially severe consequences. Ensuring compliance, or detecting non-compliance, with applicable PPE protocols is therefore an ongoing concern.

For instance, a worker may have access to the proper PPE items to mitigate safety risks, but may nonetheless improperly use a PPE item in a non-compliant and therefore risky way. In the case of a faceshield for example, a user may temporarily remove his or her faceshield in a hazardous location, and present much risk in doing so without necessarily realizing it, or forget to put the faceshield on at the required point of the procedure. Such incidents are very difficult to detect in order to allow an overseer of management of the facility to take proactive steps such as discipline or additional training for affected workers that are violating PPE protocols. Likewise, in the case of a postionable faceshield that is selectively operable in an “up” position away from one's face or a “down” position covering one's face, the faceshield may inadvertently be in the wrong position (i.e., up instead of down) when performing a hazardous task, again presenting risk without the worker necessarily realizing it. Such incidents too tend to be very difficult to detect, and management therefore generally lacks opportunity to take appropriate actions to address concerning compliance issues, especially for workers performing tasks alone.

While a protocol for some procedures require a group of persons to perform tasks together such that any PPE non-compliance can be witnessed and reported by another worker, this is not always a reliable safeguard. Different workers may approach compliance issues from various perspectives that render compliance assessment subjective rather than objective. Certain workers may be reluctant to report, or may failure to recognize or understand that a compliance violation had actually occurred. In a rarer case, a worker or a group of workers may knowingly disregard aspects of a protocol that they do not appreciate.

Unless reliably and consistently detected, intentional or unintentional violations of PPE protocols may occur indefinitely to undermine important safety considerations, and across a number of workers in different areas performing different tasks the challenges to oversee PPE compliance and detect non-compliance are multiplied. Some smart, computer-implemented monitoring systems exist in the industrial realm that intelligently incorporate sensors in items of PPE to create a greater degree of situational awareness of worker safety across groups of workers, but known systems of this type generally lack a focus on evaluating specific PPE compliance issues of the type described above.

The onset of the global “COVID-19” pandemic has raised new concerns and demands for the proper use of PPE and compliance with PPE protocols in environments that prior to COVID-19 were generally not considered “hazardous” in a manner that demonstrated a prior need for PPE. Such environments include areas of industrial facilities that are isolated from conventionally defined hazards, healthcare facilities and areas of healthcare facilities that were not previously considered to present high risk scenarios, elementary schools, middle schools, high schools, colleges and universities, offices and businesses of all types, shops and retail establishments, dining establishments, churches, entertainment venues, etc. Desirable PPE items are therefore prolifically present in these environments, but still subject to improper or non-compliant use in ways that are difficult to predict or control.

In the COVID-19 era, individual personal wellness is an important consideration to ensure that no transmission of the virus occurs to nearby persons. In general, persons have COVID-19 symptoms are strongly advised not to interact with other persons, but in some instances a person may have symptoms without necessarily realizing it. Temperature checks upon entry to an area are sometimes conducted as a course filter for screening purposes for personal entry to a space where other persons are present, but such temperature checks are limited in important aspects. Persons who passed the temperature check upon entry may develop a fever or other symptoms after the temperature check was made. In certain cases COVID-19 illness or other illnesses may rapidly develop and may suddenly impair a person considerably, so early detection of symptoms can be important but are unfortunately rare. These considerations may be of particular importance for persons that happen to be operating in a conventionally hazardous environment when a debilitating illness or health condition occurs. Existing COVID-19 protocols and electronic tools are generally reactive by nature rather than being proactive in such aspects.

Social distancing and masking are other another important consideration to address risks posed by other persons possibly having the COVID-19 virus or other conditions that can be contagiously spread or communicated to others. Faceshields may suffice for the mask requirement, but for the reasons above are subject to misuse that can defeat the virus protection desired. Some rudimentary proximity sensing and contact tracing technologies have emerged to monitor social distancing aspects and collect information that may be helpful to maintain an outbreak of illness, but they are disadvantaged in some aspects for certain hazardous environments. For instance, smart-phone based contact tracing apps are of no aid in environments wherein smart phones are prohibited. Known contact tracing apps also operate independently of PPE systems and lack capability to assess wellness in a proactive manner.

For the reasons above, improved proximity detection and monitoring systems are needed to more intelligently address compliance with protocols that are COVID-19 related and non-COVID-19 related but nonetheless implicate important wellness and PPE compliance concerns to varying degrees.

Example processor-based sensor systems are described herein that include embedded sensor technology in wearable devices, such as wearable PPE devices. Combinations of sensors are provided in intelligent wearable PPE items worn by different persons to be monitored. The intelligent wearable PPE items may be configured to connect and communicate with one another in a population of persons wearing the intelligent PPE items and also to a remote centralized system that aggregates data for review, analysis, and oversight or individual personal wellness and compliance issues in an objective and reliable manner allowing proactive management of health and safety risks in a community of persons.

Further, the processor-based systems described herein may be implemented in more general devices, such as smartphones, and other devices that a user may wear or otherwise carry on his or her person. Accordingly, although at least some implementations described herein include wearable PPE, in other embodiments, the systems and methods for detecting and analyzing proximity of one or more users can equally be implemented on a variety of non-PPE devices, such as, but not limited to, smartphones and other non-PPE devices.

The combination of sensors provided in conjunction with wearable and other electronic devices are operable in combination to provide signal inputs that may be processed and analyzed to collectively assess proximity of at least one person in relation to another person, provide feedback indicators to sensed parameters to persons wearing the devices, record contact tracing information, and output data and information to a remote device that can be accessed by overseers via informational dashboard displays. Proactive steps may be taken by overseers to quickly and proactively respond to detected issues to minimize risks presented to a community of persons wearing or carrying the devices.

In a contemplated example, a computing device may be positioned in a space, such as within a room, and used to determine a proximity, or distance, between the device and one or more other devices. Accordingly, to determine a distance between the devices, the space within which the devices are positioned by be acoustically mapped, such as based upon a comparison of an ambient acoustic signal received by the devices within the space. These mappings may facilitate, as described herein, improved selection of best fit parameters for one or more distance model equations, which may be implemented to determine distances (e.g., social distances) between persons based upon one or more wireless signal characteristics, such as received signal strength of a Bluetooth signal.

Accordingly, in at least some embodiments, the system may receive and process one or more audio signals, such as on a processor of a computing device carried on the person of an employee within an environment or area occupied by a group of such employees.

The audio data may be filtered and analyzed to obtain an environmental fingerprint, which may include one or more audio characteristics associated with the given area or environment. The environmental fingerprint for the given area may, in addition, be compared with a plurality of environmental fingerprints contained in database of predefined such fingerprints, and one or more similar environmental fingerprints may be selected from the database.

The selected environmental fingerprints may, in turn, be associated with distance model calibration parameters, which are pre-stored in a database. These distance model calibration parameters may be variously processed (e.g., as part of a linear interpolation) to obtain a set of best fit calibration parameters, whereupon the best fit calibration parameters may be supplied to a distance model equation, such as a free space path loss equation.

During operation, a wireless signal, such as a Bluetooth signal, a Wifi signal, an infrared signal, a near field communication (NFC) signal, and/or any other radio frequency (RF) or other wireless communication signal, may be received and filtered by a computing device that includes the calibrated distance model. Specifically, one or more parameters derived from the Bluetooth signal, such as RSSI, may be provided to the calibrated distance model to obtain a distance between the computing device receiving the Bluetooth signal and the Bluetooth device that transmits the signal.

As a result, a distance between persons, such as person in a work area or work environment, may be obtained. The distance calculation may, in addition and as described herein, be dramatically improved by the selection of appropriate distance model parameters based upon a preliminary analysis of the sound characteristics (e.g., audio data) within the work environment. Specifically, analysis of the audio data may facilitate selection of distance model parameters best tailored to the dimensions and other characteristics of the work area likely to impact propagation of electromagnetic energy, including Bluetooth and other radio frequency signals, within the work area or work environment.

This process may be performed substantially in real-time and for any number of such devices within the space to obtain distances between each of the devices. As a result, proximity between various devices, and thus between users of the devices, may be determined, and social distancing protocols may be monitored as desired.

In some embodiments, proximity and social distancing data may be provided to a backend monitoring system, which may display the data in an intuitive way for review by an individual responsible for ensuring workplace safety and compliance with social distancing protocols. Further, although in some embodiments, proximity detection between a variety of devices, each configured for proximity detection, is described, in at least some embodiments, proximity detection may be performed by any device equipped for proximity detection and any other device that emits a wireless signal, such as a Bluetooth signal.

In at least some embodiments, the distance model used to process a wireless signal may be based, at least in part, upon a Received Signal Strength Indication (RSSI) associated with surrounding or nearby transmitters. For instance, a selected distance model may receive, as at least one input, an RSSI of at least one nearby transmitting device, whereupon a distance between transmitting device and the receiving proximity detection device may be determined.

Based on such RSSI considerations, when persons who are determined to be distanced by less than a predetermined amount (e.g., six feet), output signals may be generated by the processor of one or more of these devices to provide feedback signals to warn each person of a proximity violation that they can quickly correct. Proximity violation information may also be recorded by each device to provide effective contact tracing when needed. In addition, as described herein, proximity information can be provided to a backend portion of the system, such as a server, which may be configured to display, or control an interconnected computing terminal to display, social distancing information as appropriate, including any warnings that are generated, as described herein.

The sensor and monitoring system described herein may be equally applicable to any of the areas listed above, or other areas that present similar issues or concerns, which are deemed hazardous in a non-conventional way solely because of COVID-19 issues or other pandemic or epidemic outbreaks that compel a use of PPE and proximity detection, and/or other areas deemed hazardous in a conventional way due to risks such as shock, blasts, impact, fire, explosion, chemical burns, and all sorts of undesirable exposure to potentially harmful elements.

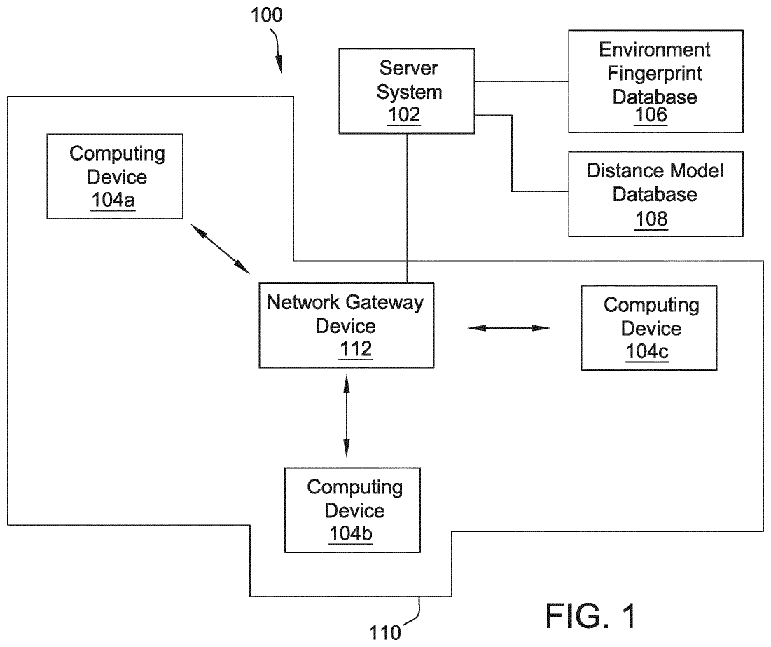

FIG. 1 is a schematic illustration of an example architecture of a health and safety monitoring system 100. The system 100, as shown, includes a remote server 102 in communication with computing devices 104a-c, such as via a network gateway device 112. System 100 may also include an environmental fingerprint database 106, and a distance model database 108. In the example embodiment, a computing device 104a-c may include a wearable electronic device, or an electronic device wearable in conjunction with (e.g., attached to) an item of PPE (e.g., a headband and faceshield device, a mask, a suit, or any other suitable PPE).

However, in at least some embodiments, a computing device 104a-c may include any of a variety of devices capable of emitting and/or receiving a wireless signal, such as a Bluetooth signal. For instance, in some embodiments, a computing device 104a-c may include a smartphone, a smart watch, a tablet computing device, and/or any other similar device that may be carried or transported on the person of a user.

In the illustrated embodiments, one remote server 102 is shown. However, in other embodiments, there are multiple remote servers 102 communicatively coupled together (e.g., in a fog computing or “cloudlet” environment). Further, in the illustrated embodiment, three proximity detection devices 104a-c are illustrated. However, in other embodiments, system 100 includes any number of such devices 104a-c.

In some embodiments, computing devices 104a-c may be enclosed in a housing and configured to be clipped, attached, or otherwise coupled to a clothing item, including a PPE item, such as a suit, a headband and/or faceshield, of a user. For example, computing devices 104a-c may be clipped or attached to a shirt, pants, a faceshield, and the like. Similarly, computing devices 104a-c may, in at least some embodiments, be attached to a lanyard and/or a similar device, such as a badge reel, and worn on the person of a user.

Each computing device 104a-c may, in the illustrated embodiment, be physically positioned on the person of a user, as described, within an area 110, such as within a room and/or an outdoor area. Area 110 may include any of a variety of dimensions and/or objects, which may affect the way wireless signals, such as Bluetooth signals, travel and reflect within area 110. More particularly, the signal strength of many wireless signals, such as ultra-high frequency radio waves (e.g., Bluetooth) can vary substantially as a result of the dimensions of area 110, such as, for example, as a result of reflections, interferences, obstacles, and the like.

As a result, accurately estimating distances between computing devices 104a-c can, in at least some embodiments, require knowledge of the environment (e.g., area 110) within which computing devices 104a-c are positioned. Further, users of computing devices 104a-c may move from one area of area 110 to another and/or within area 110. As a result of these and other factors, a variety of ambient noise and other acoustic signals may travel and reverberate within area 110.

Accordingly, as described in additional detail below, health and safety management system 100 may receive one or more acoustic signals traveling within area 110 to determine an acoustic pattern associated within area 110. These acoustic signals may be used to select or identify an acoustic pattern, or “fingerprint,” associated with area 110 most representative of area 110. As used herein, an “environmental fingerprint,” an “environmental pattern,” an “acoustic fingerprint,” or an “acoustic pattern” may refer to one or more characteristics of a pattern of sound or an acoustic signal received by a computing device 104a-c.

As a result, system 100 may identify an environment (e.g., area 110) by receiving and/or reading one or more ambient audio or acoustic signals and comparing these signals to a precomputed or predetermined group of acoustic or environmental fingerprints. A distance model may use parameters tuned to the closest one or more environmental fingerprints identified, and a distance estimation algorithm may be dynamically selected and/or adjusted to the environment (e.g., area 110) for improved accuracy in determining or estimating distances.

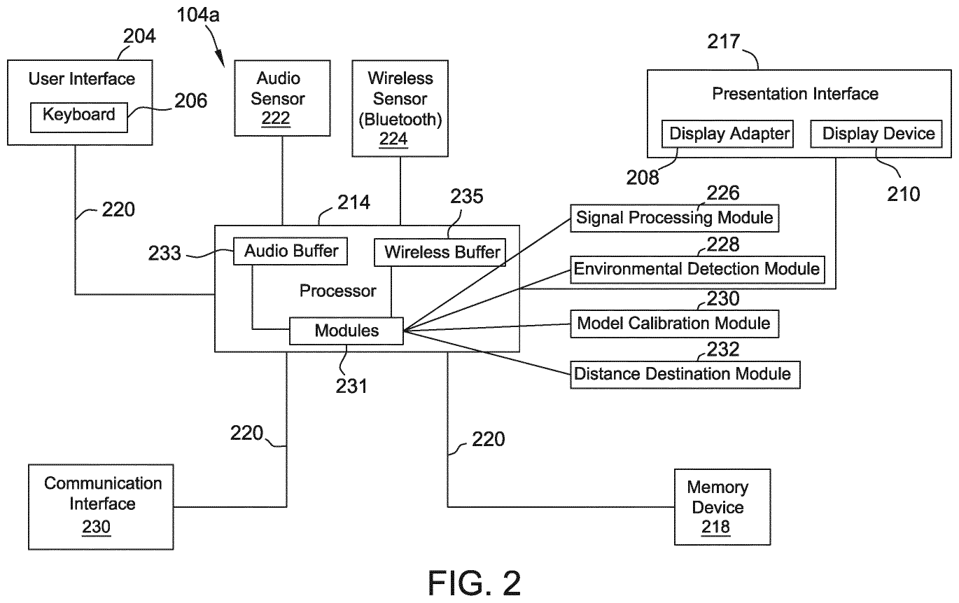

FIG. 2 is a block diagram of an example computing device 104a. In the example embodiment, computing device 104a includes a user interface 204 that receives at least one input from a user. The user interface 204 may include a keyboard 206 and/or another suitable input mechanism (e.g., a software interface, such as a graphical user interface, or “GUI”) that enables the user to input pertinent information. The user interface 204 may also include, for example, a pointing device, a mouse, a stylus, a touch sensitive panel (e.g., a touch pad and a touch screen), a gyroscope, an accelerometer, a position detector, and/or an audio input interface (e.g., including a microphone).

In the example embodiment, computing device 104a includes a presentation interface 217 that presents information, such as input events and/or validation results, to the user. The display interface 217 may also include a display adapter 208 that is coupled to at least one display device 210. More specifically, in the example embodiment, the display device 210 may be a visual display device, such as a liquid crystal display (LCD), a light-emitting diode (LED) display, an “electronic ink” display, and the like. Alternatively, the display interface 217 may include an audio output device (e.g., an audio adapter and/or a speaker) and/or a printer.

The computing device 104a also includes a processor 214 and a memory device 218. The processor 214 is coupled to the user interface 204, the display interface 217, and the memory device 218 via a system bus 220. In the example embodiment, the processor 214 communicates with the user, such as by prompting the user via the display interface 217 and/or by receiving user inputs via the user interface 204.

The term “processor” refers generally to any programmable system including systems and microcontrollers, reduced instruction set computers (RISC), complex instruction set computers (CISC), application specific integrated circuits (ASIC), programmable logic circuits (PLC), and any other circuit or processor capable of executing the functions described herein. The above examples are example only, and thus are not intended to limit in any way the definition and/or meaning of the term “processor.”

In the example embodiment, the memory device 218 includes one or more devices that enable information, such as executable instructions and/or other data, to be stored and retrieved. Moreover, the memory device 218 includes one or more computer readable media, such as, without limitation, dynamic random access memory (DRAM), static random access memory (SRAM), a solid state disk, and/or a hard disk. In the example embodiment, the memory device 218 stores, without limitation, application source code, application object code, configuration data, additional input events, application states, assertion statements, validation results, and/or any other type of data. The computing device 104a, in the example embodiment, may also include a communication interface 230 that is coupled to the processor 214 via the system bus 220. Moreover, the communication interface 230 is communicatively coupled to data acquisition devices.

In operation, a processor, such as processor 214, executes computer-executable instructions embodied in one or more computer-executable components stored on one or more computer-readable media 218 to implement aspects of the disclosure described and/or illustrated herein. However, in alternative and/or additional embodiments, server system 102 may implement any portion of the process described herein in combination with processor 214 and/or in place of processor 214. As a result, the processes for distance estimation, as described herein, may be variously performed by any of the computing devices 104a-c and/or server system 102.

The order of execution or performance of the operations in embodiments of the disclosure illustrated and described herein is not essential, unless otherwise specified. That is, the operations may be performed in any order, unless otherwise specified, and embodiments of the disclosure may include additional or fewer operations than those disclosed herein. For example, it is contemplated that executing or performing a particular operation before, contemporaneously with, or after another operation is within the scope of aspects of the disclosure.

Computing device 104a may also include one or more sensors, such as, for example, an audio sensor 220 and/or a wireless sensor 224. In the example embodiment, audio sensor 220 may include any sensor capable of receiving and/or detecting an audio or acoustic signal, such as a microphone or any other sound detecting device. In at least some embodiments, audio sensor 220 is arranged to detect sound in a frequency range between about 16 Hz to 20 Hz. Likewise, audio sensor 220 may be configured to detect sound in an ultrasonic and/or another suitable range. However, in other embodiments, these ranges may be expanded or reduced as desired.

Wireless sensor 224 may include any sensor capable of receiving and/or detecting a wireless electromagnetic signal, such as a Bluetooth signal, a Wifi signal, a radio frequency (RF) signal, and the like. In the example embodiment, wireless sensor 224 detects wireless signals emitted by one or more other proximity computing devices 104b-c. However, wireless sensor 224 can detect wireless signals output by other devices that transmit in the electromagnetic spectrum, irrespective of whether these devices are themselves capable of the proximity detection features described herein.

In the example embodiment, processor 214 may be programmed by encoding an operation using one or more executable instructions and providing the executable instructions in the memory device 218. For example, in at least some embodiments, processor 214 is programmed to determine one or more distances between one or more persons, such as one or more persons within area 110.

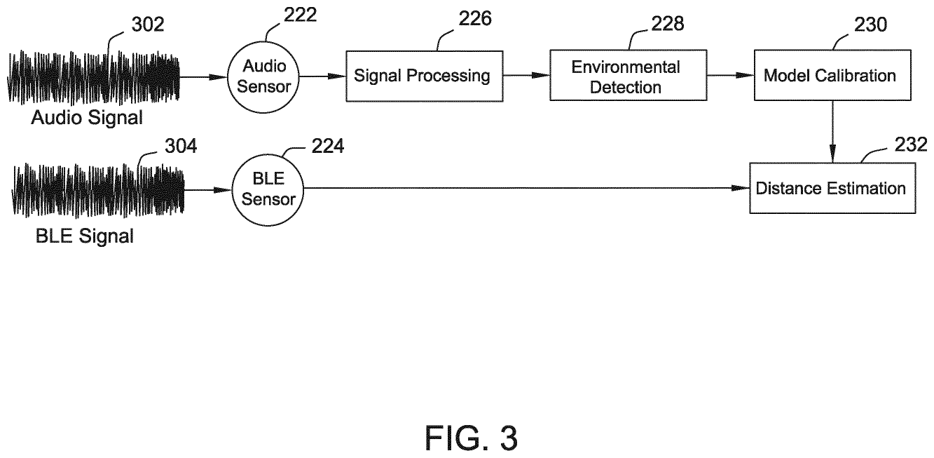

Accordingly, as shown with reference now to FIGS. 2 and 3, in at least some embodiments, the processor 214 may be programmed to execute instructions associated with one or more software modules 231, such as a signal processing module 226, an environment detection (or “fingerprinting”) module 228, a model calibration module 230, and/or a distance estimation module 232.

In various embodiments, the signal processing module 226 may filter (or “clean”) one or more acoustic signals 302 to eliminate or reduce disturbances and/or anomalies. More particularly, one or more audio signals 302, such as sound received by computing device 104a, may be read into a digital audio buffer 233 in a suitable interval, such as in a five minute interval. However, other intervals may also be implemented as desired.

In at least some embodiments, a portion of the audio signal 302 stored in the digital audio buffer 233 may be selected and/or extracted from the buffer 233 for processing. The portion may additionally be selected as desired. In at least some embodiments, the portion selected for processing may include an initial portion of the buffer 233, a central or middle portion of the buffer 233, and/or a final portion of the buffer 233. In at least one example, the final thirty seconds of the audio data associated with audio signal 302 stored to the buffer 233 are selected for processing. However, it will be appreciated that a variety of buffer 233 intervals or portions may be selected for processing.

To process the audio data extracted from the buffer 233, a voice and anomaly filter may be initially applied to the data to remove or reduce outliers and other unwanted data, such as, for example machine sounds (e.g., ambulance and first responder sounds, microwave sounds, printer sounds), and the like. The result of the initial filtering process may, in at least some embodiments, include a first filtered audio signal that includes a mostly environment-based audio signal (e.g., an audio signal from which machine sounds and other outlier sounds have been scrubbed).

The first filtered audio signal may, in at least some embodiments, be further processed in the signal processing module 226 to extract one or more features of the underlying signal, such as, for example, temporal, frequency, and/or statistical features. These features may include reverberation characteristics, squared short-term energy, Shannon entropy, standard deviation, and the like. These features may, in some cases, be referred to herein as a second filtered audio signal, or as described above, an “environmental fingerprint,” an “environment pattern,” an “acoustic fingerprint,” or an “acoustic pattern.”

As a result, the second filtered audio signal may include a feature set of extracted characteristics, which may be designated Xa, and which may correspond to the environmental fingerprint. In at least some instances, the environmental fingerprint may be influenced most predominantly by reverberations (e.g., echo) in the environment, such as based upon the dimensions and material construction of area 110. Likewise, certain machine equipment, such as a microwave oven or a computer printer, may also influence the environmental fingerprint.

In the example embodiment, the environmental fingerprint may be provided to the environment detection module 228, which may identify at least one predefined environmental fingerprint (e.g., a predefined acoustic pattern) based upon a comparison of the environmental fingerprint generated by the signal processing module 226 to a plurality of predefined environmental fingerprints stored in the environmental fingerprint database 106.

To this end, environmental fingerprint database 106 may include a collection of predefined environmental fingerprints, which may be designated Xe,i, where i∈{1, . . . , N} is the environment index of N prerecorded or predetermined environments. To selectively identify one or more predefined environmental fingerprints, the environmental fingerprint, Xa, may be compared with one or more predefined environmental fingerprints Xe,i, using, for example, a similarity search.

For example, one or more extracted characteristics of Xa may be compared to corresponding characteristics of one or more predefined environmental fingerprints Xe,i to selectively identify the predefined environmental fingerprints most similar to Xa. As a result, in at least some embodiments, one or more most similar predefined environmental fingerprints are selected from the collection of predefined environmental fingerprints Xe,i. Specifically, in at least one embodiment, a top three most similar environmental fingerprints are selected. The selected environmental fingerprints may be represented by the set: (Xa,t1, Xa,t2, Xa,t3).

Following selection of the set of most similar environmental fingerprints, processor 214 may implement the model calibration module 230, which may function to select an appropriate calibrated distance model and/or identify one or more calibration or distance model parameters according to the one or more most similar environmental fingerprints, such as (Xa,t1, Xa,t2, Xa,t3).

More particularly, in at least some embodiments, one or more parameters of at least one distance model, M, may be selected and/or identified. For example, the selected environmental fingerprints (Xa,t1, Xa,t2, Xa,t3) may be associated with appropriate model calibration parameters (e.g., in distance model database 108), and these parameters may be fed into one or more distance models.

Additionally or alternatively, the selected environmental fingerprints (Xa,t1, Xa,t2, Xa,t3) may be associated with one or more pre-calibrated or pretrained distance models, and the pretrained distance models corresponding to the selected environmental fingerprints (Xa,t1, Xa,t2, Xa,t3) may be chosen. Further, in some embodiments, one or more parameters of the selected environmental fingerprints (Xa,t1, Xa,t2, Xa,t3) may be linearly interpolated to obtain a set of final model parameters, which may be fed into a distance model equation, as described below.

Further, in at least some embodiments, a distance model may include at least a portion of the free space path loss equation, which is: RSSI=−10n log₁₀(D/D₀)+C₀. That is, in at least some embodiments, the free space path loss equation may form at least a portion of a distance model, M, and calibration parameters corresponding to the environmental fingerprints (Xa,t1, Xa,t2, Xa,t3) may be selected and input for the distance model, M, as described above.

In the free space path loss equation, D represents a distance, such as a distance between a proximity computing device 104a-c and a source of electromagnetic radiation received by the proximity computing device 104a-c, which may include a Bluetooth signal emitted by another computing device 104a-c and/or another wireless device, as described herein. Further, in the free space path loss equation, RSSI represents a received signal strength indicator associated with the wireless signal, and C₀, D₀, and n are constants representative of environmental parameters.

In various embodiments, other models that provide a mapping (such as a linear or non-linear mapping between RSSI and D) for the distance model, M: RSSI→D, may be considered and/or used as well. For example, in some embodiments, a neural network, a support vector regression, and/or another suitable model may be implemented to provide a mapping between RSSI and D.

Accordingly, in the example embodiment, a pretrained distance model may be selected from distance model database 108 for each of the one or more environmental fingerprints (Xa,t1, Xa,t2, Xa,t3), determined as described above, using the model calibration parameters. For example, for each of the environmental fingerprints (Xa,t1, Xa,t2, Xa,t3), a corresponding distance model may be selected from database 108 using the calibration parameters. Additionally or alternatively, in some embodiments, the appropriate model calibration parameters may be obtained from database 108 and fed into the free-space path loss equation to create one or more pretrained distance models.

In this example, three distance models are selected and/or formed, one for each of the environmental fingerprints (Xa,t1, Xa,t2, Xa,t3). The three distance models are represented by the set: (Mt1, Mt2, Mt3). Further, in some embodiments, one or more of the models (Mt1, Mt2, Mt3) may be combined to create a single combined distance model, which may allow for an improved approximation of the signal characteristics associated with environment (e.g., area 110). In at least one embodiment, this can be achieved using an ensemble of the models (Mt1, Mt2, Mt3) for the final calibrated model, M, fitted to the environment Xa.

In addition, in some circumstances, the distance model, M, may be trained or further calibrated to estimate a number of persons within an area, such as area 110. In various embodiments, the distance model, M, may be trained after this fashion to account for echo characteristics of area 110, which may change depending upon one or more factors, such as a number of persons present within area 110 at a given time. Accordingly, in at least some embodiments, the distance model, M, may be trained or further calibrated to estimate a number of persons present within area 110 (e.g., in real-time as the number of persons changes) based upon a number and/or characteristics of sounds emanating from various directions in the environment of area 110.

More particularly, in at least some embodiments, a plurality of audio sensors 222, such as multiple microphones, may be used to detect and quantify the sounds emanating from various directions within area 110. Data from the plurality of audio sensors 222 may be used to further calibrate the distance model, M, such as, for example, by adjusting an anticipated acoustic behavior of the environment based upon the collected sound data.

In the example embodiment, processor 214 may also implement distance estimation module 232, such as in real-time, to determine distances between computing devices 104a-c. In some embodiments, one or more wireless signals 304, such as Bluetooth signals, may be received and data associated therewith read into a wireless buffer 235. Processor 214 may receive data associated with wireless signal 304 from the buffer 235 and, in response, apply any of a variety of signal processing filters or algorithms, such as, but not limited to, a Bayesian filter configured to reduce signal noise. Other filters that may be applied include, but are not limited to, Kalman filters, particle filters, and the like.

The filtered wireless signal data may, in at least some embodiments, be provided to a most recently determined model, M, as described above, for a particular area 110, and an output (e.g., D in the free space path loss equation) may be determined. Likewise, in at least some embodiments, the filtered wireless signal data may be provided to each calibrated distance model in the set (Mt1, Mt2, Mt3) to obtain three measures of distance, D. These three measures of distance may be averaged, or the greatest or least distance taken. For example, in order to err on the side of caution, the smallest distance, D, output by each of the three distance models may be used to determine a distance between persons.

As a result, in at least some embodiments, a distance, D, between persons may be continuously estimated (in real-time or substantially real-time) using the data received in and read from wireless buffer 235. Moreover, if the distance estimation is inconclusive (e.g., if the three distance models disagree beyond a threshold), multiple distance estimation algorithms may be deployed to estimate distance, and the final distance estimate from each additional algorithm may be averaged to obtain a final distance estimate.

Moreover, in at least some embodiments, a distance estimate may be further processed for close proximity detection or analysis and displayed upstream, as described herein (e.g., via a cloud computing network), such as to an individual responsible for managing the health and safety of a workforce, contact tracing, ensuring appropriate social distances are maintained, and the like.

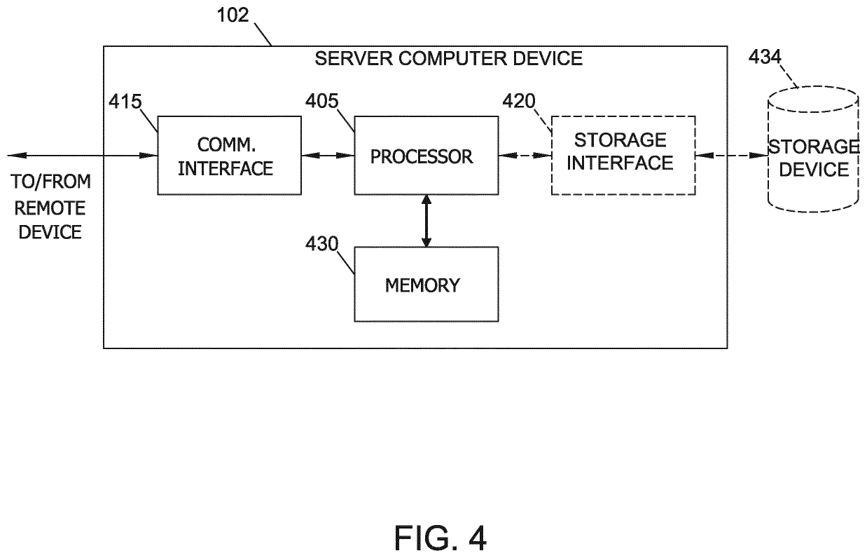

FIG. 4 illustrates an example configuration of a server system 102 (e.g., one or more server computer devices). The server computer device 102 includes a processor 405 for executing instructions. Instructions may be stored in a memory area 430, for example. The processor 405 may include one or more processing units (e.g., in a multi-core configuration).

The processor 405 is operatively coupled to a communication interface 415 such that server computer device 102 is capable of communicating with a remote device such as any of computing devices 104a-c or another server computer device 1001. For example, communication interface 415 may receive data from the computing devices 104-ac via the Internet and/or via gateway 112.

The processor 405 may also be operatively coupled to a storage device 434, such as, but not limited to, environmental fingerprint database 106 and/or distance model database 108. In some embodiments, the storage device 434 is integrated in the server computer device 102. For example, the server computer device 102 may include one or more hard disk drives as the storage device 434. In other embodiments, the storage device 434 is external to the server computer device 102 and may be accessed by a plurality of server computer devices 102. For example, the storage device 434 may include multiple storage units such as hard disks and/or solid state disks in a redundant array of inexpensive disks (RAID) configuration. The storage device 434 may include a storage area network (SAN) and/or a network attached storage (NAS) system.

In some embodiments, the processor 405 is operatively coupled to the storage device 434 via a storage interface 420. The storage interface 420 is any component capable of providing the processor 405 with access to the storage device 434. The storage interface 420 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing the processor 405 with access to the storage device 434.

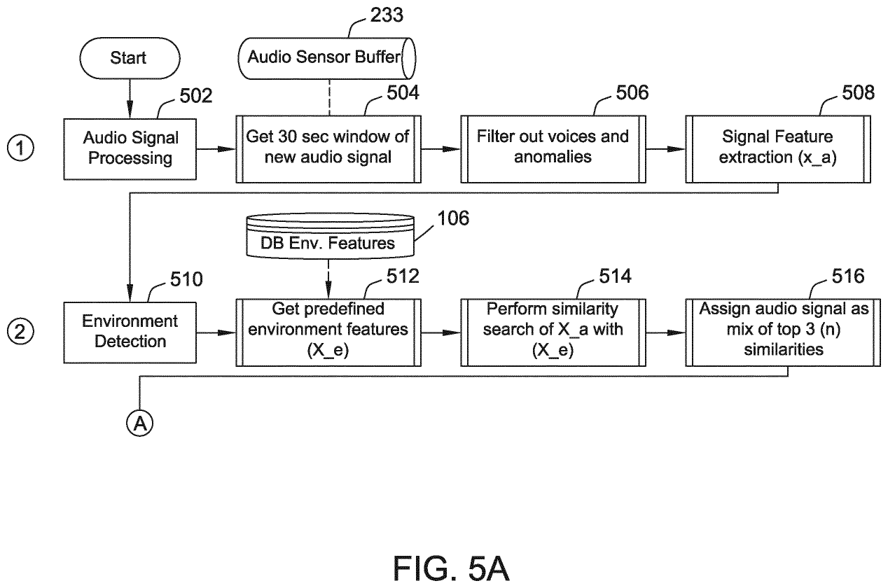

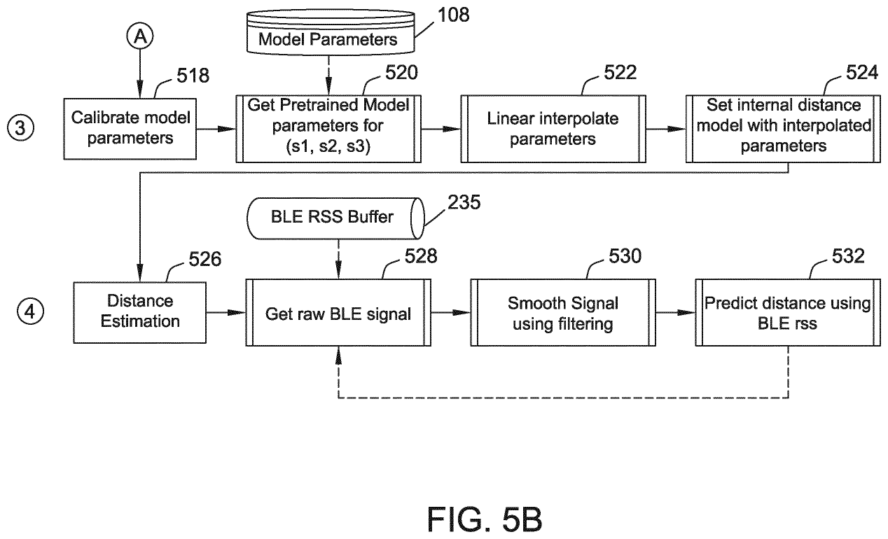

FIGS. 5A and 5B are segments of a flowchart illustrating an example embodiment of a process 500 that may be implemented by the health and safety monitoring system 100 (as shown in FIG. 1 and FIG. 3). Accordingly, in the example embodiment, and as described above, in at least some embodiments, an audio signal 302 may be received, such as by a computing device 104a and the data associated therewith stored to an audio buffer 233 (step 502). The audio data may be retrieved, in a specified time interval (such as the last thirty seconds of a five minute interval, as described above) from the audio buffer 233 for processing (step 504). In addition, in the example embodiment, anomalies and other outlier data (e.g., machine and other sounds) may be filtered from the audio data, such as by signal processing module 226, to obtain an environmental fingerprint, Xa, that includes a feature set of extracted audio characteristics (steps 506 and 508).

Next in example process 500, the environmental fingerprint, Xa, may be provided to the environment detection module 228, which may identify at least one predefined environmental fingerprint (e.g., a predefined acoustic pattern) based upon a comparison of the environmental fingerprint generated by the signal processing module 226 to a plurality of predefined environmental fingerprints stored in the environmental fingerprint database 106 (step 510).

As described herein, to selectively identify one or more predefined environmental fingerprints, the environmental fingerprint, Xa, may be compared with one or more predefined environmental fingerprints Xe,i, using, for example, a similarity search of predefined fingerprints stored in database 106 (steps 512 and 514). Following the comparison, one or more similar predefined environmental fingerprints may be selected from the collection of predefined environmental fingerprints Xe,i. Specifically, in at least one embodiment, a top three most similar environmental fingerprints are selected. The selected environmental fingerprints may be represented by the set: (Xa,t1, Xa,t2, Xa,t3) (step 516).

Following selection of the set of most similar environmental fingerprints, processor 214 may implement the model calibration module 230, which may function to select an appropriate calibrated distance model and/or identify one or more calibration or distance model parameters according to the one or more similar environmental fingerprints, such as (Xa,t1, Xa,t2, Xa,t3) (step 518).

More particularly, in at least some embodiments, one or more parameters of the selected environmental fingerprints (Xa,t1, Xa,t2, Xa,t3) may be obtained (step 520). In at least some embodiment, these parameters may be linearly interpolated to obtain a set of final model parameters (step 522). The final model parameters may, in addition, be fed into a distance model equation, such as the free space path loss equation, described above, to obtain a final distance model, M. (step 524). Further, in some embodiments, a different interpolation technique may be applied.

In response to establishing the final distance model, M, a distance estimation between a given computing device 104a-c and one or more other computing devices 104a-c may be performed (step 526). For example, processor 214 may implement distance estimation module 232, such as in real-time, to determine distances between computing devices 104a-c. In some embodiments, one or more wireless signals 304, such as Bluetooth signals, may be received and data associated therewith read into a wireless buffer 235 (step 528). Processor 214 may receive data associated with wireless signal 304 from the buffer 235 and, in response, apply any of a variety of signal processing filters or algorithms (e.g., smoothing filters), such as, but not limited to, a Bayesian filter configured to reduce signal noise (step 530).

The filtered wireless signal data may, in at least some embodiments, be provided to a most recently determined model, M, as described above, for a particular area 110, and an output (e.g., D in the free space path loss equation) may be determined, where D represents a distance between a given computing device 104a-c and at least one other computing device 104a-c (step 532).

Accordingly, a system for managing health and safety, and more particularly for determining whether adequate social distancing protocols are maintained, is described. In various embodiments, the system may receive and process one or more audio signals, such as on a processor of a computing device within an environment or area occupied by a workforce.

The audio data may be filtered and analyzed to obtain an environmental fingerprint, which may include one or more audio characteristics associated with the given area or environment. The environmental fingerprint for the given area may, in addition, be compared with a plurality of environmental fingerprints contained in database of predefined such fingerprints, and one or more similar environmental fingerprints may be selected from the database.

The selected environmental fingerprints may, in turn, be associated with distance model calibration parameters, which are pre-stored in a database. These distance model calibration parameters may be variously processed (e.g., as part of a linear interpolation) to obtain a set of best fit calibration parameters, whereupon the best fit calibration parameters may be supplied to a distance model equation, such as a free space path loss equation.

During operation, a wireless signal, such as a Bluetooth signal, may be received and filtered by a computing device that includes the calibrated distance model. Specifically, one or more parameters derived from the Bluetooth signal, such as RSSI, may be provided to the calibrated distance model to obtain a distance between the computing device receiving the Bluetooth signal and the Bluetooth device that transmits the signal.

As a result, a distance between persons, such as person in a work area or work environment, may be obtained. The distance calculation may, in addition and as described herein, be dramatically improved by the selection of appropriate distance model parameters based upon a preliminary analysis of the sound characteristics (e.g., audio data) within the work environment. Specifically, analysis of the audio data may facilitate selection of distance model parameters best tailored to the dimensions and other characteristics of the work area likely to impact propagation of electromagnetic energy, including Bluetooth and other radio frequency signals, within the work area or work environment.

The benefits and advantages of the inventive concepts are now believed to have been amply illustrated in relation to the example embodiments disclosed.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

1. An electronic device configured to estimate a distance between at least one person carrying the electronic device and at least one other person within a range of the at least one person carrying the electronic device, the electronic device comprising:
at least one audio sensor;
at least one wireless sensor;
a memory device, and
a processor configured to execute instructions stored in the memory device, which when executed by the processor, cause the processor to at least:
control the at least one audio sensor to receive at least one ambient acoustic signal captured from an environment surrounding the electronic device;
control the at least one wireless sensor to receive at least one ambient wireless signal captured from the environment;
analyze the at least one ambient acoustic signal to determine an environmental fingerprint associated with the environment, the environmental fingerprint associated with at least one acoustic characteristic;
compare the environmental fingerprint determined from the analysis of the at least one ambient acoustic signal to a database of predefined environmental fingerprints;
identify at least one predefined environmental fingerprint based upon the comparison of the environmental fingerprint to the database of predefined environmental fingerprints;
identify a pretrained distance model associated with the predefined environmental fingerprint identified from the comparison of the environmental fingerprint to the database of predefined environmental fingerprints; and
provide at least a portion of the at least one ambient wireless signal to the pretrained distance model to estimate a distance between the electronic device and a source of the at least one ambient wireless signal.

2. A method for estimating a distance between electronic devices, the method comprising:
controlling, by a processor, at least one audio sensor to receive at least one ambient acoustic signal captured from an environment;
controlling, by the processor, at least one wireless sensor to receive at least one ambient wireless signal captured from the environment;
analyzing, by the processor, the at least one ambient acoustic signal to determine an environmental fingerprint associated with the environment, the environmental fingerprint associated with at least one acoustic characteristic;
comparing, by the processor, the environmental fingerprint determined from the analysis of the at least one ambient acoustic signal to a database of predefined environmental fingerprints;
identifying, by the processor, at least one predefined environmental fingerprint based upon the comparison of the environmental fingerprint to the database of predefined environmental fingerprints;
identifying, by the processor, a pretrained distance model associated with the predefined environmental fingerprint identified from the comparison of the environmental fingerprint to the database of predefined environmental fingerprints; and
providing, by the processor, at least a portion of the at least one ambient wireless signal to the pretrained distance model to estimate a distance between the electronic device and a source of the at least one ambient wireless signal.
